(19) United States
(12) Patent Application Publication
(10) Pub. No.: US 2022/0317129 A1
(43) Pub. Date: Oct. 6, 2022

(54) METHOD FOR PREPARING BIOLOGICAL MATERIAL FOR MICROSCOPY ANALYSIS

(71) Applicants: SORBONNE UNIVERSITE, PARIS (FR); INSTITUT NATIONAL DE LA SANTE ET DE LA RECHERCHE MEDICALE, PARIS (FR); CENTRE NATIONAL DE LA RECHERCHE SCIENTIFIQUE, PARIS (FR)

(72) Inventors: ALAIN CHEDOTAL, BOURG LA REINE (FR); ROBIN VIGOUROUX, PARIS (FR)

(21) Appl. No.: 17/623,634

(22) PCT Filed: Jun. 30, 2020
(86) PCT No.: PCT/EP2020/068353

§ 371 Date: Dec. 29, 2021

(30) Foreign Application Priority Data
Jul. 1, 2019 (EP) 19305893.0

(51) Int. Cl. G01N 33/58; G01N 1/30; G01N 21/64

(52) U.S. Cl. CPC G01N 33/582; G01N 1/30; G01N 21/6458; G01N 21/6428; G01N 2021/6439; G01N 2001/305

(57) ABSTRACT

The present invention relates to a method and a kit for rendering a biological material containing melanin suitable for microscopy analysis.

### FIELD OF THE INVENTION

The present invention relates to a method and a kit for rendering a biological material containing melanin suitable for microscopy analysis.

### BACKGROUND OF THE INVENTION

The analysis of tissues for medical research, disease diagnosis, as well as the examination of tissues after in vivo or in vitro exposure to agents of interest sometimes requires the ability to examine the intact tissue in three dimensions.

For example, in the context of ocular pathologies, it may be particularly desirable to examine the eye, in its intact structure. Currently, the study of ocular pathologies in humans and in animal models is based on in vivo analyzes (such as fundoscopy) and histological analyzes. In particular, histological analyzes are performed for visualizing, through the use of biological markers (such as antibodies), different cell types within the eye's structures. Up to now, histological analyzes of the eye are carried out on histological sections or on dissected retina that is “flattened” before being visualized under a microscope. The presence of melanin in the retinal pigment epithelium makes it impossible to visualize the entire eye under the microscope. That is why, existing methods rely on removing the retinal pigmented epithelium by dissecting the retina, before observation under the microscope.

Said methods implementing histological sections and dissected retina are not satisfactory since they do not maintain the three-dimensional structure of the eye. Moreover, they require a dissection step which destroys fragile tissues (such as blood vessels). Thus, these methods are not suitable for properly “mapping” the cellular structures of the eye.

Methods of “clearing” and imaging transparent organs have been developed, in order to analyze intact organs, in three-dimensions (3D). Upon clearing, the entire organ is rendered transparent allowing the imaging light to travel end-to-end to produce high-resolution images of the unsectioned organ using a laser scanning microscope such as a multi photon or light-sheet microscope.

However, none of the existing clearing methods can target melanin-containing tissues. In particular, imaging through the eye has never been possible.

There is thus a need in a new method enabling three-dimensional microscopy analysis of the eye or other biological materials containing melanin.

### SUMMARY OF THE INVENTION

The present invention provides a method for rendering a biological material containing melanin, suitable for microscopy analysis, preferably for 3-dimension microscopy analysis.

It is herein described a method for rendering a biological material suitable for microscopy analysis, comprising the steps of:
(a) providing a biological material containing melanin;
(b) depigmenting the biological material of step (a), comprising the steps of:
   (b1) contacting the biological material with a hydrogen peroxide solution;
   (b2) irradiating the biological material obtained in step b1) with a white light or UV-visible light, wherein the biological material is in contact with the hydrogen peroxide solution; and
(c) clearing the biological material obtained by step (b), comprising the step of contacting the biological material with a clearing solution.

In particular, said biological material comprising melanin may comprise or may be a tissue or organ selected in the group consisting of: the eye, the skin, the brain (such as substantia nigra, locus coeruleus or striatum), the adrenal gland or the stria vascularis of the inner ear, preferably an eye, wherein the biological material is preferably an intact tissue or organ that maintains its natural tridimensional structure.

In a particular embodiment, steps (b1) and (b2) are cyclically repeated until the biological material is fully depigmented, and wherein the hydrogen peroxide solution in step (b1) is renewed for each cycle. The hydrogen peroxide solution may be a solution of hydrogen peroxide in alcohol having from 1 to 3 carbon atoms (such as methanol, ethanol or isopropanol), preferably methanol, and water, the concentration of hydrogen peroxide in the hydrogen peroxide solution being preferably from 3% (w/v) to 30% (w/v), preferably from 6% (w/v) to 30% (w/v), preferably from 7% (w/v) to 15% (w/v). Further, the light is preferably a warm white light and more preferably having a power around 11 W.

In a particular embodiment, step (b) further comprises, prior to step (Ill), a step of dehydrating the biological material. In particular, the dehydrating step comprises contacting the biological material with two or more successive solutions comprising an alcohol having from 1 to 3 carbon atoms, such as methanol, ethanol, or isopropanol, preferably methanol, and wherein the alcohol concentration of the successive solutions increases.

In a particular embodiment, step (b) further comprises, after step (b2), a step of rehydrating the biological material. In particular, the rehydrating step comprises contacting the biological material with two or more successive solutions comprising an alcohol having from 1 to 3 carbon atoms, such as methanol, ethanol, or isopropanol, preferably methanol, and wherein the alcohol concentration of the successive solutions decreases.

In a further particular embodiment, the clearing solution comprises ethyl cinnamate or Di-benzyl Ether.

In a particular embodiment, step (c) comprises the steps of:
(c1) dehydrating the biological material, in particular by contacting the biological material with two or more successive solutions comprising an alcohol having from 1 to 3 carbon atoms, such as methanol, ethanol, or isopropanol, preferably ethanol, and wherein the alcohol concentration of the successive solutions increases; and
(c2) clearing the dehydrated biological material of step (c1) by contacting said biological material with a clearing solution comprising ethyl cinnamate.

In another particular embodiment, step (c) comprises the steps of:
c1) dehydrating the biological material, in particular by contacting the biological material with two or more successive solutions comprising an alcohol having from 1 to 3 carbon atoms, such as methanol, ethanol, or isopropanol, preferably methanol, and wherein the alcohol concentration of the successive solutions increases;
c2) “delipidating” the biological material, in particular by contacting the biological material with one or more solutions comprising dichloromethane; and
c3) clearing the biological material by contacting the biological material with a clearing solution comprising Di-benzyl Ether.

The method herein described may further comprise a step of immunolabeling, preferably after step (b) of depigmenting the biological material and before step (c) of clearing the biological material.

It is further described a kit of parts for rendering a naturally melanin-containing biological material suitable for microscopy analysis, comprising:
a hydrogen peroxide solution, in particular a solution comprising hydrogen peroxide in alcohol having from 1 to 3 carbon atoms, such as methanol, ethanol, or isopropanol, and water, preferably methanol and water, the concentration of hydrogen peroxide in the hydrogen peroxide solution being preferably from 3% (w/v) to 30% (w/v), preferably from 6% (w/v) to 30% (w/v), preferably from 7% (w/v) to 15% (w/v);
optionally one or more solution(s) able to dehydrate and/or rehydrate the biological material, comprising an alcohol having from 1 to 3 carbon atoms, such as methanol, ethanol or isopropanol,
optionally, a washing solution, wherein the washing solution preferably comprises Phosphate Buffered Saline, in particular Dulbecco's Phosphate Buffered Saline; and
optionally, a mean providing white light or UV-visible light.

In a particular embodiment, the kit of parts may further comprise a clearing solution comprising ethyl cinnamate, preferably at a concentration of 100% (v/v).

In another particular embodiment, the kit of parts may further comprise:
one or more delipidating solution(s) comprising dichloromethane; and
a clearing solution comprising Di-benzyl Ether, preferably at a concentration of 100% (v/v).

### LEGENDS TO THE FIGURES

FIG. 1. A novel method enabling whole-eye microscopic visualization. A represents the workflow of the method: the sample is depigmented by irradiating the sample with a warm white light (3000K) at 11 W in a solution of 11% H2O2 in methanol. The sample is then cleared and subjected to light sheet fluorescent microscopy. B and B′ show images of E16 embryo heads before treatment according to the method (B) and after treatment according to the method (B′). C and C′ show images of an adult mouse eye before treatment according to the method (C) and after treatment according to the method (C′). D represents an E16 embryo head with anatomical structures. E shows an entire E16 embryo head immuno-labelled for a sensory neuron marker (TAG-1), the visual projections are manually segmented and pseudo-colored. F shows the visual projection of the E16 embryo head (retina, optic nerve, optic chiasm, and optic tract). Images B-F were obtained using “Protocol 2” as referred in the Material and Methods section, i.e. using a clearing solution of Di Benzyl Ether solution (“iDISCO+” clearing method). E,F scale bars=1000 um. Ob=olfactory bulb; Hb=Hind brain; R=rostral; C=caudal.

FIG. 2. Microscopic visualization of immunolabelled retinas after treatment according to the method of the invention. Panels A-F show adult (1 month-old) retinas depigmented and cleared according to “Protocol 2” as referred in the Material and Methods section, i.e. by using a clearing solution of Di benzyl Ether (iDisco+ clearing step). A,B show a pan retinal ganglion cell marker (RBPMS). C,D show a blood vessel marker (Collagen IV). E,F show a photoreceptor marker for short-wave opsins (OPN1sw) which is expressed in the ventral part of the retina. Panels G,H show an adult (1 month-old) eye depigmented and cleared according to “Protocol 1” of the Material and Methods, i.e. by using a clearing solution of Ethyl Cinnamate (Eci, after the depigmentation step). The eye is labeled for the pan retinal ganglion cell marker, RBPMS. A,C,G scale bars=300 um; B,D,H scale bars=100 um; E scale bar=500 um; F scale bar=150 um.

### DETAILED DESCRIPTION OF THE INVENTION

Imaging a tissue in 3-dimension is known to be challenging due to the tissue opaqueness deriving from heterogeneous optical properties among different components. Indeed, mismatched refractive indexes among different components scatters the incoming light of the microscope. In addition, in some tissues, endogenous pigments block the light from transmission, making the 3D-imaging even more complicated. This is the case of tissues that contain melanin, which is a natural pigment, characteristically brown-black, found in most organisms, including humans. The method herein described enables imaging in 3D biological material that contains melanin. Indeed, the method herein described has been shown to be effective in preparing a biological material, in particular an eye, for D3 imaging. The present method was shown to be effective, even on an entire organ, for example, an intact eye, not sectioned or sliced.

In a first aspect, it is herein disclosed a method for preparing a biological material for microscopy analysis, comprising the steps of:
(a) providing a melanin-containing biological material;
(b) depigmenting the biological material of step (a), comprising the step of irradiating the biological material with white light or UV-visible light, wherein the biological material is in contact with a hydrogen peroxide solution; and
(c) clearing the biological material obtained by step (b), comprising the step of contacting the biological material with a clearing solution.

Step (a) of Providing the Biological Material

In a particular embodiment, the melanin-containing biological material is an intact tissue or an intact organ. By “intact tissue or organ” is meant any tissue or organ whose the three-dimensional structure is preserved. In other words, the biological material maintains its natural three-dimensional structure. In particular, by “intact tissue or organ” is meant a tissue or organ that is not sectioned or sliced.

The biological material may comprise or may be any tissue or organ or tissue organoids that contains melanin. In a particular embodiment, the biological material comprises a tissue or organ selected in the group consisting of: the eye, the skin, the brain (e.g. substantia nigra, locus coeruleus or striatum), the adrenal gland or the stria vascularis of the inner ear. In addition, pathologies with hyperpigmentation, such as melanomas or melanosis (which can occur in the intestine, heart, lung, and kidney) are also suitable for this protocol. In a preferred embodiment, the biological material comprises an eye or an eye tissue. In particular, the biological material comprises an intact eye, i.e. an entire eye.

The method herein described is an ex vivo method. In other words, the biological material of interest has been extracted from the organism prior being prepared for microscopy analysis according to the method of the invention. In particular, the “organism” refers to an animal including fishes, amphibians, reptiles, birds, and mammals. Preferably, the biological material is derived from a mammal. In particular, the mammal may be laboratory animals such as mice, rats, rabbits, guinea pigs, non-human primates; pet animals such as dogs and cats; farm animals such as cows and horses; or humans. In a preferred embodiment, the animal is a laboratory animal such as mice, rats, rabbits, guinea pigs, chickens, non-human primates, preferably mice or rats. In another particular embodiment, the biological material is derived from an embryo of a laboratory animal. In particular, the biological material is derived from a mouse embryo.

In a particular embodiment, the biological material is an entire mouse embryo, for example a mouse embryo from stage E12 to E14. In another particular embodiment, the biological material is a head of mouse embryo, for example a head of mouse embryo from stage E15 to P0. In another particular embodiment, the biological material is an eye tissue or an entire eye, preferably an entire eye, from a mouse or a mouse embryo. In particular, the entire eye is an eye of a mouse from stage P0-Adult.

In the context of the present invention, the embryo, head or eye, has been extracted from the mouse or the mouse embryo, prior being prepared for microscopy analysis according to the method of the invention.

In a particular embodiment, when the biological material is a mouse eye from stage P15 to Adult, the cornea of the eye is perforated. In particular, the hole resulting from said perforation is further extended by cutting approximately ¼ of the eye circumference.

The biological material may be fixed prior to step (b) of depigmenting the biological material. Thus, a “fixation step” may be performed before step (b) of depigmenting the biological material. In this embodiment, the fixation of the biological material comprises a step of contacting the biological material with a fixation solution. In particular, the “fixation solution” is a solution comprising an aldehyde derivative compound, such as paraformaldehyde (PFA). In a particular embodiment, the concentration of PFA in the fixation solution is around 40 g/L. In particular, the biological material is contacted or immersed in a solution comprising PFA, for a sufficient period depending on the size and type of biological material. For example, the biological material is immersed in the fixation solution for 12 to 24 hours, preferably around 12 hours.

Further, after the fixation step and prior to the depigmenting step (b), the biological material may be contacted with a washing solution, for a sufficient period, for example for at least 5 minutes. This step of contacting the biological material with a washing solution may be repeated successively. For example, this step of contacting the biological material with a washing solution is performed at least 3 times. In this embodiment, the washing solution may comprise Phosphate Buffered Saline such as Dulbecco's Phosphate Buffered Saline, preferably diluted in water. In a particular embodiment, the washing solution comprises Dulbecco's Phosphate Buffered Saline and thimerosal, diluted in water. In particular, the washing solution may comprise around 10% (v/v) of 10× Dulbecco's Phosphate Buffered Saline. Further, the washing solution may comprise around 0.1 g/L of thimerosal. The biological material may be stored in this washing solution, for example for several months, prior being subjected to the depigmenting step.

In the context of the present invention, the term “contacting the biological material with a solution” means that the biological material is immersed or soaked in said solution.

In the context of the present invention, the term “around” means a relative deviation of 5 percent from the indicated value.

Step (b) of Depigmenting the Biological Material

As mentioned above, step (b) of depigmenting the biological material comprises the step of irradiating the biological material with white light or UV-visible light, wherein the biological material is in contact with a hydrogen peroxide solution. In other words, step (b) comprises, in this order, the steps of:
(b1) contacting the biological material with a hydrogen peroxide solution;
(b2) irradiating the biological material obtained in step (b1) with white light or UV-visible light, wherein the biological material is in contact with the hydrogen peroxide solution.

In a particular embodiment, the hydrogen peroxide solution comprises hydrogen peroxide in water and alcohol having from 1 to 3 carbon atoms, such as methanol, ethanol, or isopropanol, preferably methanol. The concentration of hydrogen peroxide depends on the type and size of the biological material. In a particular embodiment, the concentration of hydrogen peroxide in the hydrogen peroxide solution is from 3% (w/v) to 30% (w/v), preferably from 6% (w/v) to 30% (w/v), preferably from 7% (w/v) to 15% (w/v). In a particular embodiment the concentration of hydrogen peroxide in the hydrogen peroxide solution is about 11% (w/v).

In a particular embodiment, the light is a white light. In a further particular embodiment, the light is a warm white light. By “warm light” is meant a light source having a color temperature of around 2700-3000K, preferably around 3000K. Preferably, the light is a warm white light of 3000K.

In a particular embodiment, the light is a UV-visible light. By “UV-visible light” is meant any light having a wavelength selected in the spectra from UV light to visible light. In particular, the light may have a wavelength selected from 280 nm to 800 nm. In a particular embodiment, the light is a near UV light, preferably a near UV light having a wavelength comprised between 320 nm and 400 nm, more preferably a near UV light of around 375 nm.

In a particular embodiment, the light has a power comprised between 0.3 Watt and 15 Watt (W), preferably between 1 and 11 W. The power of the light can be adapted by the skilled person, in order to control the temperature applied to the biological material. In a particular embodiment, the light has a power around 11 W.

In a particular embodiment, the light is a white light, preferably a warm white light, having a power around 11 W.

In another particular embodiment, the light is a near UV-light, having preferably a wavelength around 375 nm, having a power around 1.3 W.

The duration of the step of irradiating the biological material depends on the type and size of the biological material and the used light. More specifically, the duration of the step of irradiating is determined by one skilled in the art so that the biological material is completely depigmented. In a particular embodiment, the duration of the step of irradiating the biological material is at least 5 hours, at least 10 hours, at least 12 hours, at least 24 hours, at least 48 hours, at least 72 hours, at least 96 hours, at least 120 hours, or at least 144 hours, preferably at least 12 hours. Preferably, the duration of the step of irradiating the biological material is around 12 hours.

In a particular embodiment, the above described steps (b1) and (b2) are cyclically repeated until the biological material is completely depigmented. By “completely depigmented” is meant that all the melanin contained in the biological material is bleached. The skilled person can easily determine if the biological material is completely depigmented by observing the biological material. In particular, the skilled person knows that the biological material is completely depigmented, once it becomes visibly white, i.e. perceptible to the eye.

The number of cycles depends on the size and type of the biological material, each “cycle” comprising one step (b1) and one step (b2). In a particular embodiment, the number of cycles is comprised between 1 and 50, between 1 and 30, between 1 and 20, between 1 and 10 or between 1 and 5.

When steps (b1) and (b2) are cyclically repeated as described above, the solution of hydrogen peroxide is preferably renewed for each cycle. By “renewed” is meant that a “fresh” hydrogen peroxide solution is used for each cycle.

In a particular embodiment, steps (b1) and (b2) are repeated every 12 hours. In this embodiment, the hydrogen peroxide solution is renewed every 12 hours.

In a particular embodiment, the biological material is agitated during step b2) of irradiating the biological material with white light or UV-visible light.

In a particular embodiment, step (b) comprises a step of dehydrating the biological material, before step (b1) of contacting the biological material with a hydrogen peroxide solution or more specifically before step (b1) of the first cycle. In a particular embodiment, the step of dehydrating the biological material comprises the step of contacting the biological material with two or more successive solutions comprising an alcohol having from 1 to 3 carbon atoms, such as methanol, ethanol, or isopropanol, wherein the alcohol concentration of the successive solutions increases. In a particular embodiment, the alcohol is methanol or ethanol, preferably methanol. The alcohol concentrations used depend on the type and size of the biological material. In an embodiment, alcohol concentrations from about 10% to about 100% (v/v), preferably from about 30% to about 100% (v/v), are used. Preferably, alcohol concentrations, in particular methanol concentrations, of about 40% (v/v), about 80% (v/v) and about 100% (v/v) are successively used, in this order. The duration of the immersion of the biological material in each solution comprising alcohol depends on the type and size of the biological material. In a particular embodiment, the biological material is contacted with each solution comprising alcohol during at least 1 hour, preferably around 1 hour and 30 minutes.

In a particular embodiment, step (b) further comprises a step of “rehydrating” the biological material, after step b2) of irradiating the biological material with white light or UV-visible light or more specifically the step b2) of the last implemented cycle. In a particular embodiment, the step of rehydrating the biological material comprises contacting the biological material with two or more successive solutions comprising an alcohol having from 1 to 3 carbon atoms such as methanol, ethanol, or isopropanol, and wherein the alcohol concentration of the successive solutions decreases. In a particular embodiment, the alcohol is methanol or ethanol, in particular methanol. The alcohol concentrations used depend on the type and size of the biological material. In an embodiment, alcohol concentrations from about 10% to about 100% (v/v), preferably from about 30% to about 100% (v/v), are used. Preferably, alcohol concentrations, in particular methanol concentrations, of about 100% (v/v), about 80% (v/v) and about 40% (v/v) are successively used, in this order. The duration of the immersion of the biological material in each solution comprising alcohol depends on the type and size of the biological material. In a particular embodiment, the biological material is contacted with each solution comprising alcohol during at least 1 hour, preferably around 1 hour and 30 minutes. Each step comprising contacting the biological material with each alcohol solution, may be repeated, i.e. the identical alcohol concentration may be used in two successive applications. For example, the step of contacting the biological material with the alcohol solution at a concentration of 100% may be repeated twice, before contacting the biological material with other alcohol solution(s) having alcohol in decreasing concentrations.

In a particular embodiment, step (b) optionally terminates by a “washing” step. In a particular embodiment, said washing step is performed after the step of rehydrating the biological material, as described above. In particular, the washing step comprises contacting the biological material with a washing solution. In this embodiment, the washing solution may comprise Phosphate Buffered Saline such as Dulbecco's Phosphate Buffered Saline preferably diluted in water. In a particular embodiment, the washing solution comprises Dulbecco's Phosphate Buffered Saline and thimerosal, diluted in water. In particular, the washing solution may comprise around 10% (v/v) of 10× Dulbecco's Phosphate Buffered Saline. Further, the washing solution may comprise around 0.1 g/L of thimerosal. The duration of this washing step depends on the type and size of the biological material. In a particular embodiment, the duration of this washing step is around 30 minutes. Said washing step may be repeated depending on the biological sample. In a particular embodiment, this washing step is repeated 2 times.

Step (c) of Clearing the Biological Material

As described above, step (c) of clearing the biological material obtained by step (b) comprises the step of contacting the biological material with a clearing solution.

By “clearing” or “optical clearing” is meant the method for making the biological material transparent. The aim of the clearing step is to equilibrate the refractive indexes of the various components of the biological material, by making it transparent. Indeed, mismatched refractive indices among different components of the biological material scatter the incoming light of the microscope. In the context of the present invention, “clearing” means eliminating refractive indices mismatch within the biological material.

Any clearing method of the state of the art may be used herein. In particular, any clearing method known to be effective on the biological material can be used. In particular, any clearing method known to be effective on a tissue or organ selected in the group consisting of: the eye, the skin, the brain (e.g. substantia nigra, locus coeruleus or striatum), the adrenal gland or the stria vascularis of the inner ear, can be used. In particular, any clearing method known to be effective for clearing an eye tissue can be used.

In a particular embodiment, the method optionally comprises an “embedding step” prior to the clearing step. In particular, an “embedding step” is carried out if the biological material is smaller than around 0.5 cm. In particular, this “embedding step” comprises embedding the biological material in an “embedding gel”. For example, the embedding gel can be an agarose gel. Methods for embedding biological material are known in the art.

In a particular embodiment, the clearing step can be an “aqueous based-clearing method” or a “solvent-based clearing method”.

Non-limitative examples of aqueous-based clearing methods include: the “SeeDB” method (Ke et al., 2013), the “Scale” method (Hama et al., 2011; 2015), the “Fruit” method (Hou et al., 2015), the “CLARITY” method (Chung et al., 2013; Tomer et al., 2014), the “PACT/PARS” method (Yang et al., 2014; Treweek et al., 2015), the “FlyClear” method (Pende et al., 2018), the “ClearT” method (Kuwajima et al., 2013) or the “SWITCH” method (Murray et al., 2015). Non-limitative examples of solvent-based clearing methods include: the “Murray's clear” method (Spalteholz, 1914), the “BABB” method (Dodt et al., 2007), the “3DISCO” method (Ertürk et al., 2011; 2012), the “iDISCO+” method (Renier et al., 2016) the “uDISCO” method (Pan et al., 2016), the “vDISCO” method (Cal et al., 2019), the “FDISCO” method (Qi et al., 2019), the “SDISCO” method (Hahn et al., 2019), the “ECi” method (Klingberg et al., 2017), or the “2ECi” method (Meixner et al., 2019).).

Preferably, the clearing method is the “iDISCO+” method (Renier et al., 2016), the “3DISCO” method (Ertürk et al., 2012) or the “ECi” method (Klingberg et al., 2017).

In a particular embodiment, the clearing solution comprises ethyl cinnamate or Di-benzyl Ether.

1) Example of a Solvent-Based Clearing Method (“Eci” Method):

The following embodiment is an example of an aqueous-based clearing method. In this embodiment, step (c) of clearing the biological material comprises the step of contacting the biological material with a clearing solution, wherein the clearing solution comprises ethyl cinnamate.

In a particular embodiment, step (c) comprises, in this order, the steps of:
(c1) dehydrating the biological material; and
(c2) clearing the dehydrated biological material by contacting said biological material with a clearing solution comprising ethyl cinnamate.

In a particular embodiment, the dehydrating step c1) comprises contacting the biological material with two or more successive solutions comprising an alcohol having from 1 to 3 carbon atoms such as methanol, ethanol, or isopropanol, wherein the alcohol concentration of the successive solutions increases. In a particular embodiment, the alcohol is methanol or ethanol, preferably ethanol. The alcohol concentrations used depend on the type and size of biological material. In an embodiment, alcohol concentrations from about 10 to about 100% (v/v) are used, preferably from about 30% to about 100% (v/v). Preferably alcohol concentrations, in particular ethanol concentrations, of about 30%, about 50%, about 70% and about 100% are successively used, in this order.

The treatment steps with a certain alcohol concentration may be repeated, i.e. the identical alcohol concentration may be used in two successive applications. For example, the step of contacting the biological material in the alcohol solution at a concentration of 100% may be done twice.

The duration of the dehydrating step and in particular the time per single application step depends on the type and size of the biological material. In a particular embodiment, the biological material is incubated during at least 1 hour, preferably around 1 hour and 30 minutes in each dehydrating solution containing alcohol. Preferably, the step c1) of dehydrating the biological material is carried out away from light. In a particular embodiment, the biological material is agitated during step c1). Preferably, the step c1) of dehydrating the biological material is carried out at room temperature.

Step c2) of clearing the dehydrated biological material comprises the step of contacting the biological material with a clearing solution comprising ethyl cinnamate. Preferably, the clearing solution comprises ethyl cinnamate at a concentration of 100% (v/v). Step c2) may be repeated, depending on the size and type of the biological material. In a particular embodiment, step c2) is repeated once, i.e. step c2) is performed twice. The duration of step c2) of clearing the dehydrated biological material depends on the type and size of the biological material. In a particular embodiment, the biological material is contacted with the clearing solution during at least 2 hours.

2) Example of Another Solvent-Based Clearing Method (“iDISCO+” Method):

The following embodiment is an example of a solvent-based clearing method. In this embodiment, step (c) of clearing the biological material comprises the step of contacting the biological material with a clearing solution, wherein the clearing solution comprises Di-benzyl

Ether.

In a particular embodiment, step (c) comprises, in this order, the steps of:
c1) dehydrating the biological material;
c2) “delipidating” the biological material;
c3) clearing the dehydrated biological material by contacting the biological material with a clearing solution comprising Di-benzyl Ether.

In a particular embodiment, the dehydrating step c1) comprises contacting the biological material with two or more successive solutions comprising an alcohol having from 1 to 3 carbon atoms, such as methanol, ethanol, or isopropanol, and wherein the alcohol concentration of the successive solutions increases. In a particular embodiment, the alcohol is methanol or ethanol, preferably methanol. The alcohol concentrations used depend on the type and size of biological material. In an embodiment, alcohol concentrations from about 10% to about 100% (v/v), preferably from 20% to about 100% (v/v), are used. Preferably, alcohol concentrations, in particular methanol concentrations of about 20% (v/v), about 40% (v/v), about 60% (v/v), about 80% (v/v) and about 100% (v/v) are successively used, in this order.

The treatment steps with a certain alcohol concentration may be repeated, i.e. the identical alcohol concentration may be used in two successive applications. For example, the step of contacting the biological material with the alcohol solution at a concentration of 100% may be done twice.

The duration of the dehydrating step and in particular the time per single application step depends on the type and size of the biological material. In a particular embodiment, the biological material is contacted in each dehydrating solution containing alcohol during at least 1 hour, preferably around 1 hour and 30 minutes. Preferably, the step c1) of dehydrating the biological material is carried out away from light. In a particular embodiment, the biological material is agitated during step c1). Preferably, the step c1) of dehydrating the biological material is carried out at room temperature.

In a particular embodiment, step c2) of “delipidating” the biological material comprises contacting the biological material with a solution comprising dichloromethane. In a particular embodiment, the step of delipidating the biological material comprises:
contacting the biological material with a first solution comprising dichloromethane diluted in methanol, preferably at a concentration of dichloromethane around 66% (v/v), and
contacting the biological material with a second solution comprising dichloromethane at a concentration of 100% (v/v).

The duration of the step of delipidating depends on the type and size of the biological material Preferably, the duration of the incubation in the first solution is around 12 hours. Preferably, the duration of the incubation in the second solution is around 30 minutes.

Step c3) of clearing the biological material comprises the step of contacting the biological material with a clearing solution comprising Di-benzyl Ether. Preferably, the clearing solution comprises Di-benzyl Ether at a concentration of 100% (v/v). Step c3) may be repeated, depending on the size and type of the biological material. In a particular embodiment, step c3) is repeated once, i.e. step c3) is performed twice. The duration of step c3) of clearing the biological material depends on the type and size of the biological material. In a particular embodiment, the biological material is contacted with the clearing solution comprising Di-benzyl Ether during at least 2 hours.

Step of Labeling the Biological Material

The method herein described may optionally comprise a step of labeling the biological material. Said labeling step may be performed after step (b) of depigmenting the biological material and preferably before step (c) of clearing the biological material.

The aim of the labeling step is to mark the biological material with a labeling reagent for identifying particular components of the biological material, such as cells or proteins. This labeling step is carried out before the observation step under the microscope.

The step of labeling the biological material may comprise the use of fluorescent label(s) and/or non-fluorescent chemical stain(s). In a particular embodiment, the labeling step comprises the use of fluorescent label(s). In a particular embodiment, the labeling step is a step of immunolabeling. “Immunolabeling” is also referred as ICC (immunocytochemistry) and IHC (immunohistochemistry). Immunolabeling enables to visualize a molecular or cellular component of interest using a combination of a specific antibody as well as a means of detection, called a tag, that is covalently linked to the antibody. Typical tags include: a fluorescent compound, gold beads, a particular epitope tag, or an enzyme that produces a colored compound. The association of the tags to the target via the antibodies provides for the identification and visualization of the antigen of interest in its native location in the tissue, such as the cell membrane, cytoplasm, or nuclear membrane. Under certain conditions the method can be adapted to provide quantitative information.

In a particular embodiment, the labeling step comprises:
a step of contacting the biological material with a composition comprising a primary antibody, i.e. the antibody that specifically binds to the molecular or cellular target of interest; and
a step contacting the biological material with a composition comprising a secondary antibody, i.e. the antibody that specifically binds to the primary antibody. The secondary antibody is designed to have a fluorophore or enzyme complex attached to it for the purposes of visualization.

In a particular embodiment, the compositions comprising the primary antibody or the secondary antibody further comprises Donkey normal serum, Triton X100 and Di-methyl sulfoxide, diluted in a solution of PBS/thimerosal. In particular, the composition comprising the primary antibody or the secondary antibody comprises 5% (v/v) of Donkey normal serum, 0.5% (v/v) of Triton X100 and 20% (v/v) of Di-methyl sulfoxide.

Step of Imaging the Biological Material

The method herein described may comprise, as the latest step, a step of imaging the biological material. In particular, the method herein described is for 3D imaging.

The imaging step can be performed with any type of optical microscope. For example, the imaging step can be performed by employing a three-dimensional microscopy technique. Preferably, the imaging step is performed by employing a confocal laser microscope, a light-sheet microscope, or a multi-photon excitation type (generally, two-photon excitation type) optical microscopy technique.

In particular, during the imaging step, the biological sample is immersed in the clearing solution as defined above. In a particular embodiment, the clearing solution comprises ethyl cinnamate or Di-benzyl Ether.

Another aspect of the invention relates to a kit of parts, for preparing a biological material containing melanin suitable for microscopy analysis, comprising:
a hydrogen peroxide solution;
optionally, one or more solutions able to dehydrate and/or rehydrate the biological material;
optionally, a washing solution; and
optionally, a mean providing white light or UV-visible light.

The kit of parts may comprise any combination of the above elements. In a particular embodiment, the kit of parts comprises a hydrogen peroxide solution, one or more solutions able to dehydrate and/or rehydrate the biological material, optionally a washing solution; and optionally a mean providing white light or UV-visible light. In another particular embodiment, the kit of parts comprises a hydrogen peroxide solution, a mean providing white light or UV-visible light, optionally one or more solutions able to dehydrate and/or rehydrate the biological material, and optionally a washing solution.

In a more particular embodiment, the kit of parts comprises all the above elements, i.e. a hydrogen peroxide solution, one or more solutions able to dehydrate and/or rehydrate the biological material, a washing solution and a mean providing white light or UV-visible light.

In a particular embodiment, the hydrogen peroxide solution comprises hydrogen peroxide in water and alcohol having from 1 to 3 carbon atoms, such as methanol, ethanol, or isopropanol, preferably methanol. The concentration of hydrogen peroxide depends on the type and size of the biological material. In a particular embodiment, the concentration of hydrogen peroxide in the hydrogen peroxide solution is from 3% (v/v) to 30% (w/v), 6% (v/v) to 30% (w/v), preferably from 7% (w/v) to 15% (w/v). In a particular embodiment the concentration of hydrogen peroxide in the hydrogen peroxide solution is about 11% (w/v).

In a particular embodiment, the one or more solution(s) able to dehydrate and/or rehydrate the biological material correspond to solution(s) of alcohol having from 1 to 3 carbon atoms, such as methanol, ethanol or isopropanol, preferably methanol. In a particular embodiment, the solution(s) correspond to a series of solutions having gradual concentrations of alcohol. The alcohol concentrations of the dehydrating/rehydrating solutions depend on the type and size of the biological material. In a particular embodiment, the alcohol concentration of the two or more dehydrating/rehydrating solutions ranges from about 10% to about 100% (v/v), in particular from about 30% to about 100% (v/v). In a particular embodiment, the kit comprises a series of 3 solutions, each having a concentration of alcohol, preferably methanol, of about 40% (v/v), about 80% (v/v) and about 100% (v/v). In another particular embodiment, the kit comprises one alcohol solution having preferably a concentration of alcohol of 100%, enabling the preparation of a series of alcohol solutions having gradual concentrations, as defined above.

The kit optionally comprises a “washing solution”, wherein said washing solution comprises Phosphate Buffered Saline, in particular Dulbecco's Phosphate Buffered Saline, preferably diluted in water. In a particular embodiment, the washing solution comprises Dulbecco's Phosphate Buffered Saline and thimerosal, diluted in water. In particular, the washing solution may comprise around 10% (v/v) of 10× Dulbecco's Phosphate Buffered Saline. Further, the washing solution may comprise around 0.1 g/L of thimerosal.

In a particular embodiment, the light is a white light. In a further particular embodiment, the light is a warm white light. By “warm light” is meant a light source having a color temperature of around 2700-3000K, preferably around 3000K. Preferably, the light is a warm white light of 3000K.

In a particular embodiment, the light is a UV-visible light. By “UV-visible light” is meant any light having a wavelength selected in the spectra from UV light to visible light. In particular, the light may have a wavelength selected from 280 nm to 800 nm. In a particular embodiment, the light is a near UV light, preferably a near UV light having a wavelength comprised between 320 nm and 400 nm, more preferably a near UV light of around 375 nm.

In a particular embodiment, the light has a power comprised between 0.3 Watt and 15 Watt (W), preferably between 1 and 11 W. The power of the light can be adapted by the skilled person, in order to control the temperature applied to the biological material. In a particular embodiment, the light has a power around 11 W.

In a particular embodiment, the light is a white light, preferably a warm white light, having a power around 11 W.

In another particular embodiment, the light is a near UV-light, having preferably a wavelength around 375 nm, having a power around 1.3 W.

In a particular embodiment, the kit of parts for preparing a biological material containing melanin for microscopy analysis, comprises:
a hydrogen peroxide solution, comprising preferably hydrogen peroxide in water and alcohol, preferably methanol, the concentration of hydrogen peroxide in the hydrogen peroxide solution being preferably from 3% (w/v) to 30% (w/v), preferably from 7% (w/v) to 15% (w/v);
optionally one or more solution(s) comprising an alcohol having from 1 to 3 carbon atoms such as methanol, ethanol or isopropanol, preferably one solution having an alcohol concentration of 100% or a series of two or more alcohol solutions having gradual concentrations of alcohol, wherein the concentration of alcohol ranges from about 30% to about 100% (v/v), preferably a series of 3 alcohol solutions, each having an alcohol concentration of about 40%, about 80% and about 100%;
optionally a “washing solution”, wherein said washing solution preferably comprises Dulbecco's Phosphate Buffered Saline, in particular around 10% (v/v) of 10× Dulbecco's Phosphate Buffered Saline; and
optionally a mean providing white light or UV-visible light, preferably white warm light having a power around 11 W.

The kit of parts may comprise any combination of the above elements. In a particular embodiment, the kit of parts comprises all the above elements.

In a particular embodiment, the kit of parts for preparing a biological material containing melanin suitable for microscopy analysis further comprises a clearing solution able to make the biological material transparent. In particular, the clearing solution comprises ethyl cinnamate or Di-benzyl Ether.

In a first particular embodiment, the clearing solution comprises ethyl cinnamate. Preferably, the clearing solution comprises ethyl cinnamate at a concentration of 100% (v/v).

In this particular embodiment, the kit of parts may further comprise one or more solutions able to dehydrate the biological material, wherein the one or more solutions correspond to solution(s) of alcohol having from 1 to 3 carbon atoms such as methanol, ethanol, or isopropanol In particular, the alcohol is methanol or ethanol, preferably ethanol. In a particular embodiment, the solution(s) correspond to a series of solutions having gradual concentration of alcohol. The alcohol concentrations of the dehydrating solutions depend on the type and size of the biological material. In a particular embodiment, the alcohol concentration, preferably the ethanol concentration, of the two or more dehydrating solutions ranges from about 10% to about 100% (v/v), preferably from about 30% to about 100% (v/v). Preferably, the kit comprises 4 solutions of alcohol, preferably ethanol, each having an alcohol concentration of about 30%, about 50%, about 70% and about 100%. In another particular embodiment, the kit comprises one alcohol solution having preferably a concentration of alcohol of 100%, enabling the preparation of a series of alcohol solutions having gradual concentrations.

Thus, in a particular embodiment, the kit of parts for rendering a biological material containing melanin suitable for microscopy analysis, comprises:
one or more alcohol solutions comprising an alcohol having from 1 to 3 carbon atoms such as methanol, ethanol or isopropanol, preferably one solution having an alcohol concentration of 100% or a series of two or more alcohol solutions having gradual concentrations of alcohol, wherein the concentration of alcohol ranges from about 30% to about 100% (v/v), preferably a series of 3 alcohol solutions, each having an alcohol concentration of about 40%, about 80% and about 100%;
a hydrogen peroxide solution, comprising preferably hydrogen peroxide in water and methanol, more preferably hydrogen peroxide in the hydrogen peroxide solution is from 3% (w/v) to 30% (w/v), preferably from 6% (w/v) to 30% (w/v), preferably from 7% (w/v) to 15% (w/v);
optionally a “washing solution”, wherein said washing solution preferably comprises Dulbecco's Phosphate Buffered Saline, in particular around 10% (v/v) of 10× Dulbecco's Phosphate Buffered Saline;
a series of alcohol solutions, in particular ethanol solutions, wherein the concentration of alcohol ranges from about 30% to about 100% (v/v), preferably a series of 4 alcohol solutions, each having an alcohol concentration of about 30%, about 50%, about 70% and about 100%; and
a clearing solution comprising ethyl cinnamate, preferably at a concentration of 100% (v/v).

In a second particular embodiment, the clearing solution comprises Di-benzyl Ether. Preferably, the clearing solution comprises Di-benzyl Ether at a concentration of 100% (v/v).

In this particular embodiment, the kits of parts may further comprise one or more delipidating solution. In a particular embodiment, the delipidating solution(s) comprise dichloromethane. In a particular embodiment, the kit of parts comprises a first delipidating solution comprising dichloromethane diluted in methanol, preferably at a concentration of dichloromethane around 66% (v/v). In a particular embodiment, the kit of parts comprises a second delipidating solution comprising dichloromethane at a concentration of 100% (v/v).

In addition, in this particular embodiment, the kit of parts may further comprise one or more solutions able to dehydrate the biological material. In a particular embodiment, the one or more solution(s) able to dehydrate and/or rehydrate the biological material correspond to solution(s) of alcohol having from 1 to 3 carbon atoms, such as methanol, ethanol or isopropanol, preferably methanol. In a particular embodiment, the solution(s) correspond to a series of solutions having gradual concentrations. The alcohol concentrations of the dehydrating solutions depend on the type and size of the biological material. In a particular embodiment, the alcohol concentration of the series of dehydrating solutions ranges from about 20% to about 100% (v/v). Preferably, 5 solutions of alcohol, preferably methanol, are used, each having an alcohol concentration of about 20%, about 40%, about 60%, about 80% and about 100%. In another particular embodiment, the kit comprises one alcohol solution having a concentration of alcohol of 100%, enabling the preparation of a series of alcohol solutions having gradual concentrations.

Thus, in a particular embodiment, the kit of parts for rendering a biological material containing melanin suitable for microscopy analysis comprises:
one or more alcohol solutions comprising an alcohol having from 1 to 3 carbon atoms such as methanol, ethanol or isopropanol, preferably one solution having an alcohol concentration of 100% or a series of two or more alcohol solutions having gradual concentrations of alcohol, wherein the concentration of alcohol ranges from about 30% to about 100% (v/v), preferably a series of 3 alcohol solutions, each having an alcohol concentration of about 40%, about 80% and about 100%;
a hydrogen peroxide solution, comprising preferably hydrogen peroxide in water and methanol, more preferably hydrogen peroxide in the hydrogen peroxide solution is from 3% (w/v) to 30% (w/v), preferably from 6% (w/v) to 30% (w/v), preferably from 7% (w/v) to 15% (w/v);
optionally a “washing solution”, wherein said washing solution preferably comprises Dulbecco's Phosphate Buffered Saline, in particular around 10% (v/v) of 10× Dulbecco's Phosphate Buffered Saline;
a series of alcohol solutions, in particular methanol solutions, wherein the concentration of alcohol ranges from about 20% to about 100% (v/v), preferably a series of 5 alcohol solutions, each having an alcohol concentration of about 20%, about 40%, about 60%, about 80% and about 100%;
one or more one or more delipidating solution(s), in particular a first delipidating solution comprising dichloromethane diluted in methanol, preferably at a concentration of dichloromethane around 66% (v/v), and a second delipidating solution comprising dichloromethane at a concentration of 100% (v/v);
a clearing solution comprising Di-benzyl Ether, preferably at a concentration of 100% (v/v), and
optionally a mean providing white light or UV-visible light, preferably white warm light having a power around 11 W.

The kit of parts of the invention may further comprise a notice detailing the method of use the solutions comprised therein according to the method of the invention.

It is also described the use of the kit of parts, as defined above, in a method for preparing a biological material for microscopy analysis. In a particular embodiment, the kit of parts is used for rendering a biological mater al containing melanin compatible with microscopy analysis, in particular 3-D microscopy analysis.

Further aspects and advantages of the invention will be disclosed in the following experimental section, which illustrates the claimed invention.

### EXAMPLES

Materials and Methods

Solutions:

BUFFER H1 (for 1 L)

Add 100 mL of 10× Dulbecco Phosphate Buffer

Add 900 mL of distilled water (milliQ)

Using an analytical balance, weigh 0.1 g of thimerosal powder and add to the mix

BUFFER H2 (For 1 L)

Bring to boil 1 L of distilled H₂O (milliQ) under a chemical fumehood

Add 40 g of Paraformaldehyde

Add 2-3 drops of 1N NaOH

Stir, using a stirring rod, until solution is completely clear

Chill the solution on ice

Filter the solution using a Filter

Aliquot and store at −20 C

BUFFER E1 (30% Ethanol)

Add 60 mL of Ethanol

Add 140 mL of distilled H₂O (milliQ).

BUFFER E2 (50% Ethanol)

Add 100 mL of Ethanol

Add 100 mL of distilled H₂O (milliQ).

BUFFER E3 (70% Ethanol)

Add 140 ml of Ethanol

Add 60 ml of distilled H₂O (milliQ).

BUFFER E4 (100% Ethanol)

Add 200 mL of Ethanol.

BUFFER E5 (Ethyl Cinnamate)

Add 200 mL of 100% Ethyl Cinnamate in a glass bottle, protect from light.

BUFFER M20 (20% Methanol)

Add 40 mL of Methanol

Add 160 mL of 1×PBS.

BUFFER M40 (40% Methanol)

Add 80 mL of Methanol

Add 120 mL of 1×PBS.

BUFFER M3 (60% Methanol)

Add 120 ml of Methanol

Add 80 ml of 1×PBS.

BUFFER M4 (80% Methanol)

Add 160 ml Methanol

Add 40 ml of distilled H₂O

BUFFER M5 (100% Methanol)

Add 200 mL of 100% Methanol in a glass bottle, protect from light.

BUFFER D1 (1/3 Methanol 2/3 Dichloromathene)

Add 66 ml of Dichloromethane

Add 33 ml of Methanol.

BUFFER D2 (Dichloromethane)

Add 200 ml of Dicholoromethane

BUFFER D3 (Di-Benzyl Ether)

Add 200 ml of Di-benzyl Ether

BUFFER H (11% Hydrogen Peroxide in Methanol)

Add 66.6 mL of Hydrogen Peroxide

Add 133.3 mL of Methanol

Keep at 4 C away from light

BUFFER I1 (5% Donkey Normal Serum; 0.5% TritonX100 in PBS/Thimerosal)

Add 2.5 mL of Donkey Normal Serum

Add 1 mL of 25% TritonX100

Qsp with PBS Thimerosal solution (46.5 mL)

BUFFER 12 (5% Donkey Normal Serum; 0.5% TritonX100; 20% Di-methyl Sulfoxide in PBS/Thimerosal (0.1 g/L)

Add 2.5 mL of Donkey Normal Serum

Add 1 mL of 25% TritonX100

Add 10 mL of Dimethyl Sulfoxide

Qsp with PBS Thimerosal solution (36.5 mL)

BUFFER 13 (0.5% TritonX100 in PBS/Thimerosal (0.1 g/L)

Add 1 mL of 25% TritonX100

Qsp with PBS Thimerosal solution (49 mL)

Embedding Gel solution (For 100 mL) (1.5% Agarose)

Add 1.5 g of agarose

Add 100 mL of 1×TAE buffer. This can be replaced with dH2O.

Dissolve by bringing the solution to a boil using a microwave

| Product | Cat. | | Company |
|---|---|---|---|
| Paraformaldehyde | 1.04005.1000 | | Merck-Millipore |
| Sodium Hydroxide | 1.06469.1000 | | Merck-Millipore |
| (NaOH) | | | |
| 10× Dulbecco Phosphate | 14200-083 | | Gibco |
| Buffered Saline | | | |
| Thimerosal | T8784-5 | g | Sigma |
| Hydrogen Peroxide | 23613.297 | | VWR |
| Methanol | 20847.360 | | VWR |
| Ethanol | 32221-1L-M | | Sigma |
| Dichloromethane | 270997-1 | L | Sigma |
| (>99.8%) | | | |
| Benzyl Ether (98%) | 108014-1 | kg | Sigma |
| Donkey Normal Serum | S30-100 | mL | Merck-Millipore |
| Dimethyl Sulfoxide | D8418-250 | mL | Sigma |
| TritonX-100 | X100-500 | mL | Sigma |

| | | |
|---|---|---|
| Agarose | 2267.4 | Roth |
| MX TAE Buffer | 15558-026 | Invitrogen |

Protocol 1:

1. Harvesting the Eyes:
From stages E12-E14, process the entire embryo: Sacrifice the female mouse. Make an incision antero-posterior at the linea alba (midline of the abdomen). Make a second incision laterally at the most anterior side of the initial incision to expose the uterus. Using scissors, remove the uterine sac containing the embryos and place them in a 50 mL falcon tube with 15 mL of Buffer H1. Under the microscope, using two forceps, perforate a single uterine sac to remove the embryo.
From stages E15-P0: process the entire head
From stages P0-P15: remove the eye: Using a curved scissor, gently cut the connective tissue around the eye, place the curved scissors behind the eye and section the optic nerve, using forceps hold the remaining connective tissue around the eye and cut any excess tissue anchoring the eye to the eye socket.
From stage P16-Adult: Remove the eye and make a slit in the cornea (this helps with the penetration of the fixative as well as the immune-labeling). Once the eye is harvested, anchor the eye using a forceps attached to some connective tissue. Using a 30^{1/2} gauge needle, perforate the cornea of the eye. Using curved scissors, extend the hole by cutting circumferentially. ¼ of the eye circumference is sufficient.

2. Fixation (Minimum Volume: 2 mL): Perform all Steps Under a Fume Hood
a. Place the embryo/dissected eye in Buffer H2 overnight at 4 C
   i. For embryos from E12-E14, put entire embryo in 5 mL of Buffer H2
   ii. For embryos from E15-P0, put entire Head in 5 mL of Buffer H2
   iii. For dissected eye from P0-P15, put entire eye in 2 mL of Buffer H2
   iv. For dissected eye from P16-Adult, put entire eye in 2 mL of Buffer H2
b. Wash tissue 3× in Buffer H1 (5 mins per wash)
c. Proceed with the next step. Alternatively, the tissues can be stored in Buffer H1 for several months.

3. Depigmentation: Perform all Steps Under a Fume Hood
a. Place the embryos/eyes in a 2 mL tube, fill with 2 mL Buffer M40 for 1 h 30 mins
b. Remove the previous solution and fill with Buffer M80 for 1 h 30 mins
c. Remove the previous solution and fill with Buffer M100 for 1 h 30 mins
   For stages E12-P0:
      i. Remove the previous Buffer and add Buffer H.
      ii. Place tubes on a rotor at 12 rpm under a warm white light >1000 W on the tube, overnight at room temperature.
   For stages P0-P7:
      i. Remove the previous buffer and add Buffer H.
      ii. Place tubes on an rotor at 12 rpm under a warm white light >1000 W on the tube, overnight at room temperature.
      iii. The next day, renew the Buffer H; place the warm white light on the tube at room temperature. Renew the solution every 12 hours.
      iv. Repeat this step for 3 days (until the eye is completely white)
   For P7-P30:
      i. Remove the previous buffer and add Buffer H.
      iii. Place tubes on an rotor at 12 rpm under a warm white light >1000 W on the tube, overnight at room temperature.
      ii. The next day, renew the Buffer H; place the warm white light on the tube at room temperature. Renew the solution every 12 hours.
      iii. Repeat this step for 6 days (until the eye is completely white)
   for mouse >1 month
      i. Remove the previous buffer and add Buffer H.
      ii. Place tubes on an rotor at 12 rpm under a warm white light >1000 W on the tube, overnight at room temperature.
      iii. The next day, renew the Buffer H; place the warm white light on the tube at room temperature. Renew the solution every 12 hours.
      iv. Repeat this step for 7-10 days (until the eye is completely white)
d. Once the eyes are completely white, remove Buffer H, and wash for 30 mins the samples with Buffer M100.
e. Repeat 2 times
f. Remove the previous buffer and add Buffer M80 for 1 h 30 mins
g. Remove the previous buffer and add Buffer M40 for 1 h 30 mins
h. Remove the previous buffer and add Buffer H1 for 30 mins; repeat 2 times

4. Immuno-Labelling (Minimum Volume: 2 mL)
a. Take a new 2 mL Eppendorf tube, place your sample and Add 2 mL of Buffer I1
b. Place tube on a rotor at 12 rpm, at room temperature for 1 day minimum.
c. Using Buffer 12, dilute your primary antibodies to the desired concentration.
d. Remove Buffer I1 and add 2 ml of Buffer 12 containing your primary antibodies.
e. Place the tube on a rotor at 12 rpm at room temperature or a minimum of 7 days.
f. Remove the Buffer 12 and add 2 mL of Buffer 13. Place the tube on a rotor at 12 rpm at room temperature.
g. Repeat this step at least 6 times with a minimum of 1 hour intervals (˜1 day).
h. The next day, Using Buffer 12, dilute your secondary antibodies to your desired concentration.
i. Take a 0.22 um filter and a syringe (appropriate for the volume), pre-wash the filter using 2 mL of Buffer 12. Using the same syringe and 0.22 um filter, add the Buffer 12 containing your secondary antibody solution into a new 2 mL Eppendorf tube.
j. Add your samples in the secondary solution, place on a rotor at 12 rpm at room temperature for a minimum of 3 days. Protect from the light.
k. Remove the Buffer 12 and add 2 mL of Buffer 13. Place the tube on a rotor at 12 rpm at room temperature. Protect from the light
l. Repeat this step at least 6 times with 1 hour intervals (˜1 day).
m. For the final wash, remove Buffer 13 and add 2 mL of Buffer H1 for 5 min. Repeat this step 2 times. Protect from the light.

5. Clearing (Minimum Volume: 5 mL)

All steps are performed at room temperature away from the light.

Embedding (in particular if the sample is smaller than ˜0.5 cm)
i. Dissolve the Embedding Gel in a microwave for 1 min 30 secs. Allow the solution to cool down
ii. Add 3 mL of the Embedding Gel in a weighing boat to create a flat platform, making sure that the entire surface is covered.
iii. Once the Embedding Gel has solidified in the weighing boat, position the eye/embryos
iv. Add 1 drop of Embedding Gel to anchor the eye/embryo, allow the solution to solidify.
v. Cover the entire embryo/eye with the remaining Embedding Gel
vi. Leave to solidify at 4 C for 30 mins.
vii. Using a razorblade, cut around your tissue (embryo/eye) leaving 0.1-0.3 cm of agarose on each side. Proceed to clearing.

Clearing
a. Place your sample into a 5 mL Eppendorf tube. Fill the tube with Buffer E1. Place the tube in a rotor at 12 rpm at room temperature for 1 h 30 mins or overnight.
b. Remove the previous solution and fill with Buffer E2. Place the tube on a rotor at 12 rpm for 1 h 30 mins at room temperature.
c. Remove the previous solution and fill with Buffer E3. Place the tube on a rotor at 12 rpm for 1 h 30 mins at room temperature.
d. Remove the previous solution and fill with Buffer E4. Place the tube on a rotor at 12 rpm for 1 h at room temperature.
e. After 1 h, refresh the Buffer E4 to assure the samples are equilibrated.
f. Remove the previous solution and fill with Buffer E5. Place the tube on a rotor at 12 rpm overnight at room temperature.
g. After 30 mins, refresh the solution with fresh Buffer E5. Allow at least 2 hours prior to imaging the tissue.

Protocol 2:

1. Harvesting the Eyes:

Same steps as protocol 1.

2. Fixation (Minimum Volume: 2 mL): Perform all Steps Under a Fume Hood

Same steps as protocol 1.

3. Depigmentation: Perform all Steps Under a Fume Hood

Same steps as protocol 1.

4. Immune-Labelling (Minimum Volume: 2 mL)

Same steps as protocol 1.

5. Clearing (Minimum Volume: 5 mL)

All steps are performed at room temperature away from the light.

Embedding (in particular if the sample is smaller than ˜0.5 cm)

Same steps as protocol 1.

Clearing
a. Place your sample into a 5 mL Eppendorf tube. Fill the tube with Buffer M20. Place the tube in a rotor at 12 rpm at room temperature for 1 h 30 mins or overnight.
b. Remove the previous solution and fill with Buffer M40. Place the tube on a rotor at 12 rpm for 1 h 30 mins at room temperature.
c. Remove the previous solution and fill with Buffer M60. Place the tube on a rotor at 12 rpm for 1 h 30 mins at room temperature.
d. Remove the previous solution and fill with Buffer M80. Place the tube on a rotor at 12 rpm for 1 h 30 mins at room temperature.
e. Remove the previous solution and fill with Buffer M100. Place the tube on a rotor at 12 rpm for 1 h at room temperature.
f. After 1 h, refresh the Buffer M100 to assure the samples are equilibrated.
g. Remove the previous solution and fill with Buffer D1. Place the tube on a rotor at 12 rpm overnight at room temperature.
h. The next day, remove the previous solution and fill the tube with Buffer D2. Place the tube on a rotor at 12 rpm for 30 mins.
i. Remove the previous solution and fill the tube with Buffer D3.
j. After 30 mins, refresh the solution with fresh Buffer D3. Allow at least 2 hours prior to imaging the tissue.

Results

The present method, using either Protocol 1 or Protocol 2 as detailed above, enabled the depigmentation and transparisation of an intact tissue containing a mouse eye, thereby rendering it compatible with 3-D microscopy observation.

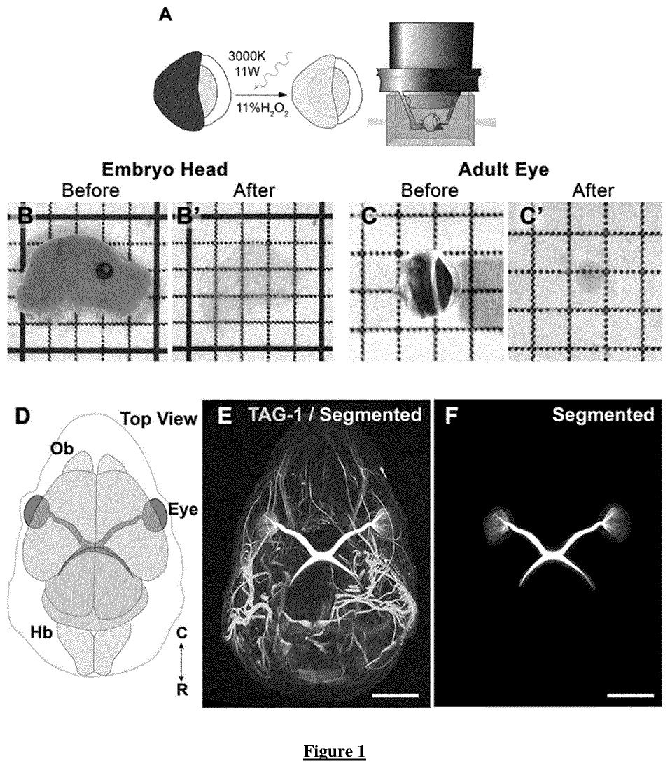

FIGS. 1B and 1B′ demonstrate that a whole mouse embryo head (stage E16) is perfectly depigmented and transparised, after treatment according to Protocol 2 as described above. Similarly, FIGS. 1C and 1C′ demonstrate that a mouse adult eye is perfectly depigmented and transparised, after treatment according to the Protocol 2 as described above. FIGS. 1E and 1F demonstrate that the method enables to visualize immuno-labelled anatomical structures of the embryo head, using light sheet fluorescent microscopy. FIGS. 1D-1F show that the method herein described did not alter anatomical structures of the head embryo.

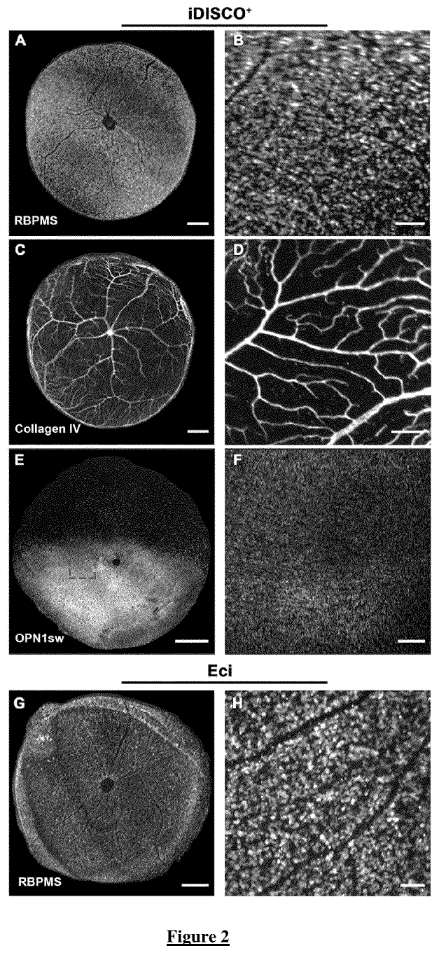

FIG. 2 relates to the microscopic visualization of immunolabelled retinas after treatment according to Protocol 1 (FIGS. 2G and 2H) and after treatment according to Protocol 2 (FIGS. 2A-2F). It can be seen that the present method enables the visualization of different immuno-labelled components of the eye, including a pan retinal ganglion cell marker (RBPMS), a blood vessel marker (Collagen IV) and a photoreceptor marker for short-wave opsins (OPN1sw). Said figures confirm that immunolabelling step was efficiently performed and not altered by the depigmentation and clearing steps of the method.

In conclusion, the method according to the invention enables efficient 3D-imaging of an eye or an eye-containing tissue, that is intact, i.e. not dissected or sliced. The cellular structures of the eye are not altered by the present method. In particular, the present method did not alter the structures, at a protein level, thus enabling efficient immuno-labelling and efficient 3D-microscopy observation.

This method is thus suitable for understanding the organization of the cellular structures of the eye, such as blood vessels or ganglion cells.

### REFERENCES

Cai, R., Pan, C., Ghasemigharagoz, A., Todorov, M. I., Förstera, B., Zhao, S., Bhatia, H. S., Parra-Damas, A., Mrowka, L., Theodorou, D., et al. (2019). Panoptic imaging of transparent mice reveals whole-body neuronal projections and skull—meninges connections. Nat. Neurosci. 22, 317-327.
Chung, K., and Deisseroth, K. (2013). CLARITY for mapping the nervous system. Nat. Methods 10, 508-513.
Dodt, H.-U., Leischner, U., Schierloh, A., Jährling, N., Mauch, C. P., Deininger, K., Deussing, J. M., Eder, M., Zieglgänsberger, W., and Becker, K. (2007). Ultramicroscopy: three-dimensional visualization of neuronal networks in the whole mouse brain. Nat. Methods 4, 331-336.
Ertürk, A., Mauch, C. P., Hellal, F., Förstner, F., Keck, T., Becker, K., Jährling, N., Steffens, H., Richter, M., Hübener, M., et al. (2011). Three-dimensional imaging of the unsectioned adult spinal cord to assess axon regeneration and glial responses after injury. Nat. Med. 18, 166-171.
Ertürk, A., Becker, K., Jährling, N., Mauch, C. P., Hojer, C. D., Egen, J. G., Hellal, F., Bradke, F., Sheng, M., and Dodt, H.-U. U. (2012). Three-dimensional imaging of solvent-cleared organs using 3DISCO. Nat. Protoc. 7, 1983-1995.
Hahn, C., Becker, K., Saghafi, S., Pende, M., Avdibašić, A., Foroughipour, M., Heinz, D. E., Wotjak, C. T., and Dodt, H. (2019). High-resolution imaging of fluorescent whole mouse brains using stabilised organic media (sDISCO). J. Biophotonics e201800368.
Hama, H., Kurokawa, H., Kawano, H., Ando, R., Shimogori, T., Noda, H., Fukami, K., Sakaue-Sawano, A., and Miyawaki, A. (2011). Scale: a chemical approach for fluorescence imaging and reconstruction of transparent mouse brain. Nat. Neurosci. 14, 1481-1488.
Hama, H., Hioki, H., Namiki, K., Hoshida, T., Kurokawa, H., Ishidate, F., Kaneko, T., Akagi, T., Saito, T., Saido, T., et al. (2015). ScaleS: an optical clearing palette for biological imaging. Nat. Neurosci. 18, 1518-1529.
Hou, B., Zhang, D., Zhao, S., Wei, M., Yang, Z., Wang, S., Wang, J., Zhang, X., Liu, B., Fan, L., et al. (2015). Scalable and DiI-compatible optical clearance of the mammalian brain. Front. Neuroanat. 9, 19.
Ke, M.-T., Fujimoto, S., and Imai, T. (2013). SeeDB: a simple and morphology-preserving optical clearing agent for neuronal circuit reconstruction. Nat. Neurosci. 16, 1154-1161.
Klingberg, A., Hasenberg, A., Ludwig-Portugall, I., Medyukhina, A., M?nn, L., Brenzel, A., Engel, D. R., Figge, M. T., Kurts, C., and Gunzer, M. (2017). Fully Automated Evaluation of Total Glomerular Number and Capillary Tuft Size in Nephritic Kidneys Using Lightsheet Microscopy. J. Am. Soc. Nephrol. 28, 452-459.
Kuwajima, T., Sitko, A. A., Bhansali, P., Jurgens, C., Guido, W., and Mason, C. (2013). ClearT: a detergent- and solvent-free clearing method for neuronal and non-neuronal tissue. Development 140, 1364-1368.
Meixner, K., Taniguchi, Y., Pasierbek, P., Knoblich, J. A., Masselink, W., Tanaka, E. M., Bonnay, F., Reumann, D., and Murawala, P. (2019). Broad applicability of a streamlined ethyl cinnamate-based clearing procedure. Development 146, dev166884.
Murray, E., Cho, J. H., Goodwin, D., Ku, T., Swaney, J., Kim, S.-Y., Choi, H., Park, Y.-G., Park, J.-Y., Hubbert, A., et al. (2015). Simple, Scalable Proteomic Imaging for High-Dimensional Profiling of Intact Systems. Cell 163, 1500-1514.
Pan, C., Cai, R., Quacquarelli, F. P., Ghasemigharagoz, A., Lourbopoulos, A., Matryba, P., Plesnila, N., Dichgans, M., Hellal, F., and Ertürk, A. (2016). Shrinkage-mediated imaging of entire organs and organisms using uDISCO. Nat. Methods 13, 859-867.
Pende, M., Becker, K., Wanis, M., Saghafi, S., Kaur, R., Hahn, C., Pende, N., Foroughipour, M., Hummel, T., and Dodt, H.-U. (2018). High-resolution ultramicroscopy of the developing and adult nervous system in optically cleared Drosophila melanogaster. Nat. Commun. 9, 4731.
Qi, Y., Yu, T., Xu, J., Wan, P., Ma, Y., Zhu, J., Li, Y., Gong, H., Luo, Q, and Zhu, D. (2019). FDISCO: Advanced solvent-based clearing method for imaging whole organs. Sci. Adv. 5, eaau8355.
Renier, N., Adams, E. L., Kirst, C., Wu, Z., Azevedo, R., Kohl, J., Autry, A. E., Kadiri, L., Umadevi Venkataraju, K., Zhou, Y., et al. (2016). Mapping of Brain Activity by Automated Volume Analysis of Immediate Early Genes. Cell 165, 1789-1802.
Spalteholz, W. (1914). Über das Durchsichtigmachen von menschlichen und tierischen Präparaten und seine theoretischen Bedingungen, nebst Anhang: Über Knochenfarbung. S. Hirzel, Leipzig.
Tomer, R., Ye, L., Hsueh, B., and Deisseroth, K. (2014). Advanced CLARITY for rapid and high-resolution imaging of intact tissues. Nat. Protoc. 9, 1682-1697.
Treweek, J. B., Chan, K. Y., Flytzanis, N. C., Yang, B., Deverman, B. E., Greenbaum, A., Lignell, A., Xiao, C., Cai, L., Ladinsky, M. S., et al. (2015). Whole-body tissue stabilization and selective extractions via tissue-hydrogel hybrids for high-resolution intact circuit mapping and phenotyping. Nat. Protoc. 10, 1860-1896.
Yang, B., Treweek, J. B., Kulkarni, R. P., Deverman, B. E., Chen, C.-K., Lubeck, E., Shah, S., Cai, L., and Gradinaru, V. (2014). Single-cell phenotyping within transparent intact tissue through whole-body clearing. Cell 158, 945-958.

1-16. (canceled)

17. A method for preparing a biological material for microscopy analysis, comprising the steps of:
(a) providing a biological material containing melanin;
(b) depigmenting the biological material of step (a), comprising the steps of:
(b1) contacting the biological material with a hydrogen peroxide solution;
(b2) irradiating the biological material obtained in step b1) with white light or UV-visible light, wherein the biological material is in contact with the hydrogen peroxide solution; and
(c) clearing the biological material obtained by step (b), comprising the step of contacting the biological material with a clearing solution.

18. The method according to claim 17, wherein steps (b1) and (b2) are cyclically repeated until the biological material is fully depigmented, and wherein the hydrogen peroxide solution in step (b1) is renewed for each cycle.

19. The method according to claim 17, wherein the hydrogen peroxide solution is a solution of hydrogen peroxide in alcohol having from 1 to 3 carbon atoms and water.

20. The method according to claim 19, wherein the concentration of hydrogen peroxide in the hydrogen peroxide solution is from 6% (w/v) to 30% (w/v).

21. The method according to claim 17, wherein the light is a warm white light.

22. The method according to claim 17, wherein step (b) further comprises, prior to step (b1), a step of dehydrating the biological material.

23. The method according to claim 22, wherein the dehydrating step comprises contacting the biological material with two or more successive solutions comprising an alcohol having from 1 to 3 carbon atoms and wherein the alcohol concentration of the successive solutions increases.

24. The method according to claim 17, wherein step (b) further comprises, after step (b2), a step of rehydrating the biological material.

25. The method according to claim 24, wherein the rehydrating step comprises contacting the biological material with two or more successive solutions comprising an alcohol having from 1 to 3 carbon atoms and wherein the alcohol concentration of the successive solutions decreases.

26. The method according to claim 17, wherein the clearing solution comprises ethyl cinnamate or di-benzyl ether.

27. The method according to claim 17, wherein step (c) comprises the steps of:
(c1) dehydrating the biological material; and
(c2) clearing the dehydrated biological material by contacting said biological material with a clearing solution comprising ethyl cinnamate.

28. The method according to claim 27, wherein step (c1) comprises contacting the biological material with two or more successive solutions comprising an alcohol having from 1 to 3 carbon atoms and wherein the alcohol concentration of the successive solutions increases.

29. The method according to claim 17, wherein step (c) comprises the steps of:
(c1) dehydrating the biological material;
(c2) delipidating the biological material; and
(c3) clearing the biological material by contacting the biological material with a clearing solution comprising di-benzyl ether.

30. The method according to claim 17, wherein said biological material comprises a tissue or organ selected in the group consisting of: the eye, the skin, the brain, the adrenal gland or the stria vascularis of the inner ear, wherein the biological material is an intact tissue or organ that maintains its natural tridimensional structure.

31. The method according to claim 17, further comprising a step of immunolabeling, after step (b) of depigmenting the biological material and before step (c) of clearing the biological material.

32. The method according to claim 17, wherein step (c) comprises the steps of:
(c1) dehydrating the biological material by contacting the biological material with two or more successive solutions comprising an alcohol having from 1 to 3 carbon atoms and wherein the alcohol concentration of the successive solutions increases;
(c2) delipidating the biological material by contacting the biological material with one or more solution(s) comprising dichloromethane; and
(c3) clearing the biological material by contacting the biological material with a clearing solution comprising di-benzyl ether.

33. A kit of parts for rendering a naturally melanin-containing biological material suitable for microscopy analysis, comprising:
a hydrogen peroxide solution;
optionally one or more solution(s) able to dehydrate and/or rehydrate the biological material, comprising an alcohol having from 1 to 3 carbon atoms;
optionally, a washing solution; and
optionally, a means providing white light or UV-visible light.

34. The kit of parts according to claim 33, further comprising a clearing solution comprising ethyl cinnamate.

35. The kit of parts according to claim 33, further comprising:
one or more delipidating solution(s) comprising dichloromethane; and
a clearing solution comprising di-benzyl ether.

36. The kit of parts according to claim 33, wherein the hydrogen peroxide solution is a solution comprising hydrogen peroxide in an alcohol having from 1 to 3 carbon atoms and water.
